(19) United States
(12) Patent Application Publication
(10) Pub. No.: US 2013/0204146 A1
(43) Pub. Date: Aug. 8, 2013

(54) RESPIRATION INDUCING APPARATUS AND CONTROL METHOD THEREOF

(71) Applicant: Samsung Electronics Co., Ltd., Gyeonggi-do (KR)

(72) Inventor: Chul-Ho CHO, Gyeonggi-do (KR)

(73) Assignee: Samsung Electronics Co., Ltd., Gyeonggi-do (KR)

(21) Appl. No.: 13/759,635

(22) Filed: Feb. 5, 2013

(30) Foreign Application Priority Data
Feb. 8, 2012 (KR) 10-2012-0012722

(51) Int. Cl. A61B 5/00; A61B 5/0205; A61B 5/113

(52) U.S. Cl. CPC A61B 5/7246; A61B 5/742; A61B 5/7475; A61B 5/6823; A61B 5/113; A61B 5/0205; A61B 5/7278
USPC 600/484; 600/529; 600/534

(57) ABSTRACT

A respiration inducing apparatus is provided, and includes a respiration waveform data collection unit for collecting respiration waveform data from a user; a storage unit for storing a respiration waveform template; a controller for comparing the respiration waveform data and the respiration waveform template and determining a respiration fidelity based on a similarity of a comparison result; and a user interface unit for displaying at least one of the respiration waveform data, the respiration waveform template, and the respiration fidelity.

### PRIORITY

This application claims priority under 35 U.S.C. §119(a) to Korean Application Serial No. 10-2012-0012722, which was filed in the Korean Intellectual Property Office on Feb. 8, 2012, the entire content of which is incorporated herein by reference.

### BACKGROUND OF THE INVENTION

1. Field of the Invention

The present invention generally relates to a respiration inducing apparatus and a control method using respiration waveform data, and more particularly, to a respiration inducing apparatus and a control method capable of inducing respiration having a specific period and depth.

2. Description of the Related Art

It is widely known and proven that stress is a major cause of developing various diseases. Accordingly, various methods of reducing stress are being researched. Particularly, in psycho-analytical and related medical fields, respiration during a specific period is identified as a method of reducing stress.

Statistical results of stress reduction experiments in the medical field shows continuing respiration in a specific period, e.g., six times in one minute, has a stress reduction effect. The aforementioned continuous respiration in a specific period has an effect not only for stress reduction but also for a good sleep, and thus, continuous respiration in the specific period needs to be promoted.

On the other hand, there is no specific disclosure of a technique for promoting continuous respiration in a specific period. Accordingly, a method of promoting the continuous respiration in the specific period is needed. However, since a user may easily become tired of performing continuous respiration in the specific period, there is a need for an apparatus for stably promoting respiration in the specific period and a control method thereof which triggers a user's interest.

### SUMMARY OF THE INVENTION

Accordingly, an aspect of the present invention is to solve at least the above-described problems occurring in the prior art, and to provide at least the advantages described below.

Another aspect of the present invention is to provide a respiration inducing apparatus and a control method thereof capable of inducing respiration in a specific period.

In accordance with an aspect of the present invention, a respiration inducing apparatus is provided. The respiration inducing apparatus includes a respiration waveform data collection unit for collecting respiration waveform data from a user; a storage unit for storing a respiration waveform template; a controller for comparing the respiration waveform data and the respiration waveform template and determining a respiration fidelity based on a similarity of a comparison result; and a user interface unit for displaying at least one of the respiration waveform data, the respiration waveform template, and the respiration fidelity.

In accordance with another aspect of the present invention, a method of controlling a respiration inducing apparatus is provided. The method includes collecting respiration waveform data from a user; reading out a pre-stored respiration waveform template; comparing the respiration waveform data and the respiration waveform template and determining a respiration fidelity based on a similarity of a comparison result; and displaying at least one of the respiration waveform data, the respiration waveform template, and the respiration fidelity.

### BRIEF DESCRIPTION OF THE DRAWINGS

The above and other aspects, features, and advantages of the present invention will be more apparent from the following detailed description taken in conjunction with the accompanying drawings, in which:

FIG. 1 illustrates a block diagram of a respiration inducing apparatus according to an embodiment of the present invention;

FIGS. 2A and 2B illustrate conceptual views in which a user wears a respiration inducing apparatus including a respiration waveform data collection unit;

FIG. 2C illustrates respiration waveform data in analog form according to an embodiment of the present invention;

FIG. 3 illustrates a block diagram of a respiration inducing apparatus according to an embodiment of the present invention;

FIGS. 4A and 4B illustrate examples of game screens according to an embodiment of the present invention;

FIG. 5 illustrates a conceptual view of a game play screen according to an embodiment of the present invention;

FIGS. 6A and 6B illustrate conceptual views explaining a respiration fidelity and a score thereof;

FIG. 7 illustrates a flow chart of a control method of a respiration inducing apparatus according to an embodiment of the present invention;

FIG. 8 illustrates a flow chart of a control method of a respiration inducing apparatus according to another embodiment of the present invention;

FIG. 9 illustrates a flow chart of a game screen display method according to another embodiment of the present invention; and

FIG. 10 illustrates a block diagram of a respiration inducing apparatus according to another embodiment of the present invention.

### DETAILED DESCRIPTION OF EMBODIMENTS OF THE PRESENT INVENTION

Hereinafter, embodiments of the present invention will be described in detail with reference to the accompanying drawings. In the following description, the same elements will be designated by the same reference numerals wherever possible. Further, in the following description of the present invention, a detailed description of known functions and configurations incorporated herein will be omitted when it may make the subject matter of the present invention unclear.

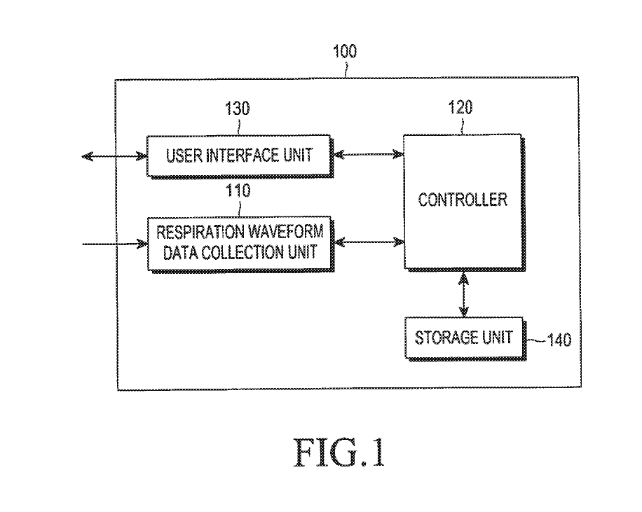

FIG. 1 illustrates a block diagram of a respiration inducing apparatus according to an embodiment of the present invention;

As shown in FIG. 1, a respiration inducing apparatus 100 includes a respiration waveform data collection unit 110, a controller 120, a user interface unit 130, and a storage unit 140. Here, the respiration inducing apparatus 100 is not limited in structure as long as the respiration inducing apparatus 100 has a structure of including the respiration waveform data collection unit 110, the controller 120, the user interface unit 130, and the storage unit 140; however, in an embodiment of the present invention, the respiration inducing apparatus 100 may be implemented in a smart phone or a tablet personal computer (PC). The smart phone or the tablet PC may include a touch screen corresponding to the user interface unit 130, a central processor corresponding to the controller 130, and a storage means corresponding to the storage unit 140.

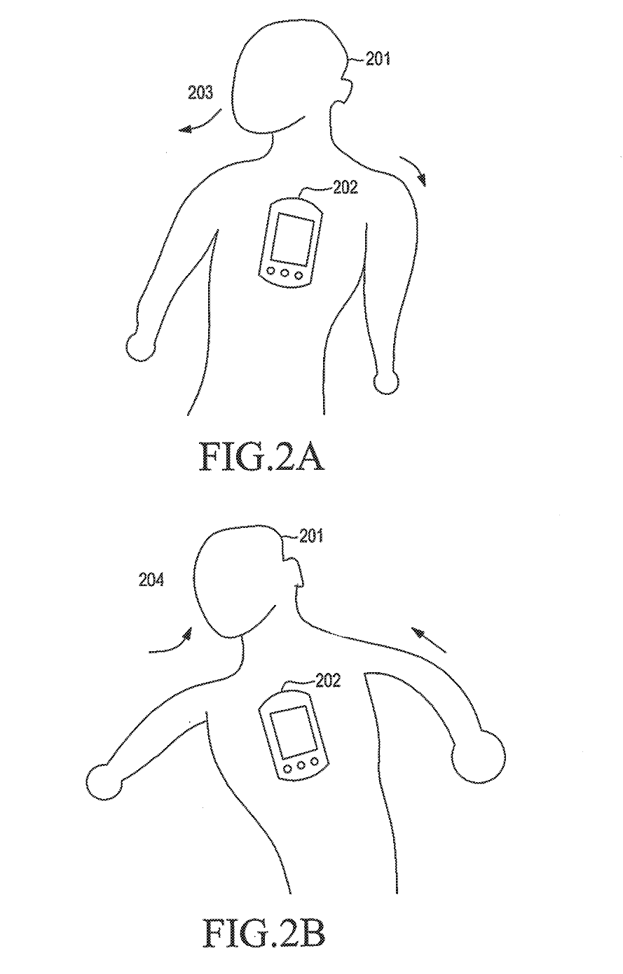

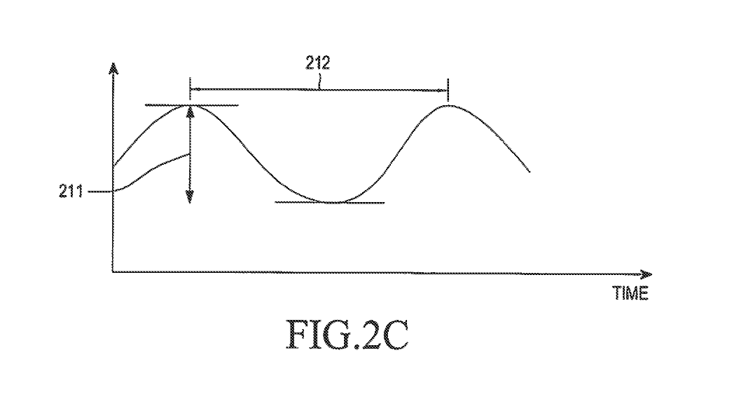

The respiration waveform data collection unit 110 collects respiration waveform data of a user who wears, directly or indirectly, the respiration inducing apparatus 100. Here, the respiration waveform data is time series data and, for example, has a higher value with respect to inhalation and a lower value with respect to exhalation. A form of the respiration waveform data will be described later in more detail with reference to FIG. 2C.

The respiration waveform data collection unit 110 collects respiration waveform data in analog form to be output to the controller 120. Alternatively, the respiration waveform data collection unit 110 may perform preset pre-treatment on the respiration waveform data in analog form to be converted into a digital form and output.

The respiration waveform data collection unit 110, for example, is implemented in a three axis acceleration sensor. Here, the three axis acceleration sensor means a sensing means capable of measuring an acceleration respectively corresponding to three dimensions of a space. The respiration waveform data collection unit 110 may be directly mounted to the user.

When the user performs respiration, the user's diaphragm moves downward when the user inhales and the diaphragm moves upward when the user exhales. The respiration waveform data collection unit 110 is preferably mounted around the level of the a user's diaphragm.

FIG. 2A illustrates a conceptual view in which the user wears the respiration inducing apparatus 100 including the respiration waveform data collection unit 110. As shown in FIG. 2A, a user 201 mounts the respiration inducing apparatus 202 near the diaphragm. As described above, when the user exhales at 203, the diaphragm moves upward.

The respiration waveform data collection unit 110 senses an acceleration of the ascending diaphragm. For example, the respiration waveform data collection unit 110 senses an increasing acceleration in an opposite direction of gravity due to the ascending of the diaphragm. Alternatively, the respiration waveform data collection unit 110 senses an increasing acceleration in a direction toward the chest because the chest ascends due to the ascending diaphragm.

FIG. 2B illustrates a conceptual view for explaining a process in which the user inhales. Similar to the embodiment of FIG. 2A, the user 201 mounts a respiration inducing apparatus 202 near the diaphragm. When the user 201 inhales at 204, the diaphragm of the user 201 moves downward.

The respiration waveform data collection unit 110 senses an acceleration of the descending diaphragm. For example, the respiration waveform data collection unit 110 senses an increasing acceleration in a direction of gravity due to the descending of the diaphragm. Alternatively, the respiration waveform data collection unit 110 senses an increasing acceleration in an opposite direction of the chest because the chest descends due to the descending diaphragm.

The respiration waveform data collection unit 110 outputs collected acceleration increase/decrease information to the controller 120 as raw data in analog form. Alternatively, as described above, the respiration waveform data collection unit 110 outputs respiration waveform data, which is converted into the digital form through predetermined pre-treatment, to the controller 120.

FIG. 2C shows respiration waveform data in analog form according to an embodiment of the present invention. As shown in FIG. 2C, the respiration waveform data may be time series data. The respiration waveform data has a certain depth 211 and a certain period 212. As a user's respiration becomes faster, the period 212 may be shortened, and as a user's respiration volume is increased, the depth 211 may be increased.

On the other hand, the respiration waveform data collection unit 110 may collect the respiration waveform data in various methods other than a method of measuring an acceleration of the user's diaphragm or chest, and this will be described in further detail later.

The controller 120 receives the respiration waveform data to be compared with a respiration waveform template read out from the storage unit 140. The respiration waveform template is respiration waveform related data including information about a period and a depth which are proven to have a stress reduction effect. For example, the respiration waveform template may be sinusoidal data having a frequency of 360 Hz.

The controller 120 compares the respiration waveform data and the respiration waveform template and outputs a comparison result to the user interface unit 130. The controller 120 determines a similarity between the respiration waveform data and the respiration waveform template and determines a user's current respiration fidelity based on the determined similarity.

For example, the controller 120 determines a correlation between the respiration waveform data and the respiration waveform template and determines a correlation value as the similarity.

Here, time series data of the respiration waveform is designated as [x1, x2, . . . , xn] and time series data of the respiration waveform template is designated as [y1, y2, . . . , yn]. In this case, a correlation r_{xy} between the respiration waveform data and the respiration waveform template may be determined by Equation (1).

$${r}_{xy} = \frac{{\sum }_{i = 1}^{n} ( {x}_{i} - \overline{x} ) ( {y}_{i} - \overline{y} ) }{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} \qquad Equation ( 1 )$$

In Equation 1, is an average of the respiration waveform data and y̅ is an average of the respiration waveform template. As the correlation value according to Equation (1) is higher, a waveform similarity between the respiration waveform data and the respiration waveform template is higher and, as the correlation value according to Equation (1) is lower, the waveform similarity between the respiration waveform data and the respiration waveform template is lower.

The controller 120 determines the above described correlation to determine the user's respiration fidelity. For example, the controller 120 determines that a current respiration fidelity is higher when the correlation is higher and determines that the current respiration fidelity is lower when the correlation is lower.

The controller 120 may render a graphic of the above described respiration fidelity in a manner which more conforms to a user's intuition. For example, the controller 120 may score the respiration fidelity to render a user-friendly graphic including the scored respiration fidelity. The controller 120 may control the user interface unit 130 to display the rendered graphic.

The controller 120 may control the user interface unit 130 to display, in addition to the respiration fidelity, a respiration induction indicator for a higher respiration fidelity, a respiration waveform data currently input, and a respiration waveform template.

The controller 120 controls an overall operation of the respiration inducing apparatus 100. The controller 120 may reads out, from the storage unit 140, and performs a program, and an application, an algorithm about the overall operation of the respiration inducing apparatus 100. The controller 120 may be implemented in a form of a central processor, a microprocessor, an integrated circuit (IC) chip, or a mini computer, and it is apparent to those skilled in the art that the controller 120 is not limited in kind. The controller 120 may further include at least one of a ROM and a RAM which are additional storage means, and in this case, the central processor, the ROM, and the RAM may be interconnected through an internal bus.

The user interface unit 130 receives an input signal from the user. The user interface unit 130 may also display the rendered graphic. The user interface unit 130 may include, for example, a key input means and a display means. On the other hand, the user interface unit 130 may be implemented in a form of a touch screen. When the user interface unit 130 is implemented in the form of the touch screen, the user interface unit 130 includes a touch screen module and a touch screen driving module at the same time.

The user interface unit 130 displays rendered graphic data input from the controller 120. The user interface unit 130 receives a user input from the user. Here, the user input may be an input which causes the overall operation of the respiration inducing apparatus 100. Particularly, the user input may be a manipulation in a form of a game, which will be described later in further detail.

As described above, the user identifies a current respiration status and an ideal respiration status of the user at the same time and receives a similarity between the current respiration status and the ideal respiration status. Accordingly, the user may change a user's respiration status into the ideal respiration status, and the ideal respiration status may be maintained.

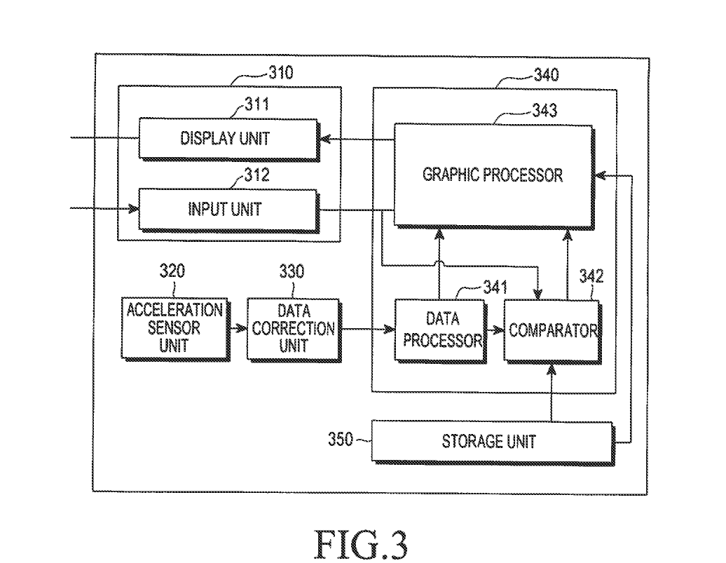

FIG. 3 illustrates a block diagram of a respiration inducing apparatus according to an embodiment of the present invention.

As shown in FIG. 3, a respiration inducing apparatus 300 includes an interface unit 310, an acceleration sensor unit 320, a data correction unit 330, a controller 340, and a storage unit 350. The user interface unit 310 includes a display unit 311 and an input unit 312. Also, the controller 340 includes a data processor 341, a comparator 342, and a graphic processor 343.

The user interface unit 310 displays the graphic rendered by the controller 340, as described above in connection with FIG. 1. Also, the user interface unit 310 receives a user input. Specifically, the display unit 311 displays the respiration waveform data and the respiration waveform template. Further, the display unit 311 displays the current respiration fidelity based on the similarity between the respiration waveform data and the respiration waveform template. Also, the display unit 311 displays a respiration induction indicator which can increase the respiration fidelity. The display unit 311 may be implemented in an LCD module and may be implemented as the touch screen together with the input unit 312. The input unit 312 receives a user input.

The acceleration sensor unit 320 may be, for example, a three axis acceleration sensor. The acceleration sensor unit 320 senses an acceleration which is applied to the respiration inducing apparatus 300. As described in connection with FIGS. 2A and 2B, acceleration information sensed by the acceleration sensor unit 320 corresponds to the respiration waveform data. The acceleration sensor unit 320 outputs sensed raw data in analog form to the data correction unit 330.

The data correction unit 330 amplifies input respiration waveform data with a preset gain. Also, the data correction unit 330 may filter the amplified respiration waveform data. Here, the data correction unit 330 includes an amplification means for amplifying with the preset gain and a filtering means for filtering. For example, the amplification means may be implemented as an OP-AMP and the filtering means may be implemented as a low pass filter, a high pass filter, or a band pass filter, or a combination thereof.

The respiration waveform data corrected by the data correction unit 330 is input to the data processor 341.

The data processor 341 performs an analog to digital conversion (ADC) on the input respiration waveform data in analog form and outputs the respiration waveform data in the digital form. Specifically, the data processor 341 samples the input respiration waveform data in analog form in a preset period. The data processor 341 performs quantification on the sampled respiration waveform data and generate the respiration waveform data in digital form based on a quantification result. The data processor 341 outputs the generated respiration waveform data in digital form to the comparator 342 and the graphic processor 343.

The comparator 342 reads out the respiration waveform template from the storage unit 350 and compares the read out respiration waveform template with the respiration waveform data. The comparator 342, for example, determines the correlation between the respiration waveform data and the respiration waveform template based on Equation (1). A comparison result of the comparator 342 corresponds to a similarity between the respiration waveform data and the respiration waveform template. Further, the similarity may be based on the current respiration fidelity.

On the other hand, the comparator 342 may compare the respiration waveform data and the respiration waveform template in various methods other than using the correlation and it should be understood by those skilled in the art that the present invention is not limited in this regard.

The graphic processor 343 renders at least one of the respiration waveform data input from the data processor 341, the respiration waveform template read out from the storage unit 350, and the current respiration fidelity input from the comparator 342 to generate graphic data. The graphic processor 343 generates rendered graphic data based on a graphic generation algorithm read out from the storage unit 350. The graphic processor 343 may be implemented in a central processor and render a graphic with respect to each pixel of the display unit 311 in a serial manner. Also, the graphic processor 343 may be implemented in a graphic processor (GPU) to render the graphic with respect to each pixel of the display unit 311 in a parallel manner.

The graphic processor 343, for example, may render a graphic of a game screen which may trigger a user's interest.

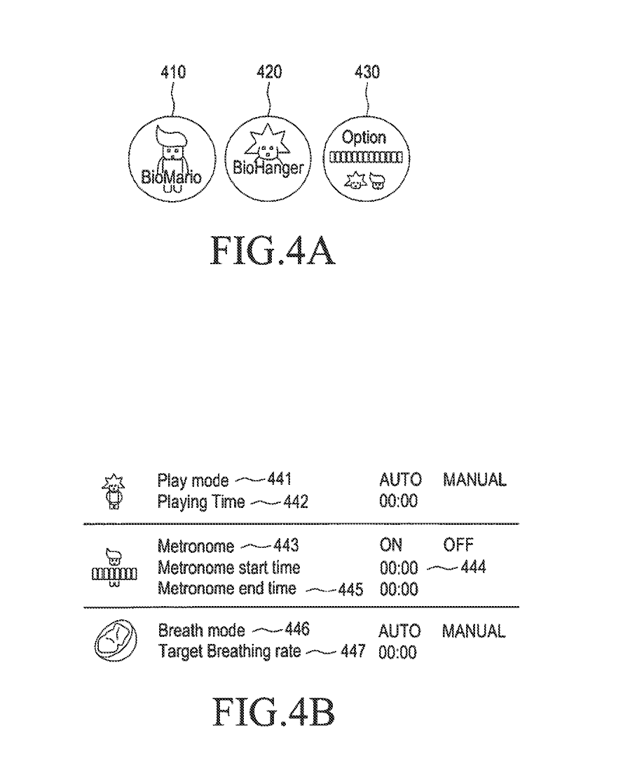

FIGS. 4A and 4B illustrate example game screens according to an embodiment of the present invention.

FIG. 4A shows a game screen related to a character which is a subject that plays a game and an option in a game according to an embodiment of the present invention. The user selects one of characters 410, 420 through manipulation of the input unit 312. Also, the user may select an option menu 430 through manipulation of the input unit 312.

FIG. 4B is a screen in which the option menu 430 in FIG. 4A is selected. The user determines whether a play mode 441 is automatic or manual through the manipulation of the input unit 312. Also, the user may adjust a play time 442 through manipulation of the input unit 312.

The user determines whether to use a metronome 443 through the manipulation of the input unit 312. Here, the metronome is an example of the respiration inducing indicator and is a means for indicating a predetermined time period based on a period of the respiration waveform template. This will be described below in further detail.

The user may adjust a start time 444 and an end time 445 of the metronome through the manipulation of the input unit 312.

The user determines a respiration mode 446 through the manipulation of the input unit 312. The respiration mode 446 may be about whether to use a pre-stored respiration waveform template or whether to use a respiration waveform template adjusted by the user.

The user may adjust a period 447 of the respiration waveform template through the manipulation of the input unit 312. The graphic processor 343 adjusts and renders the period of the respiration waveform template when a user manipulation is involved.

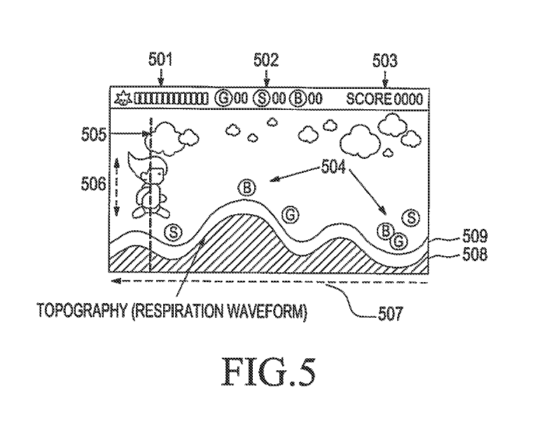

FIG. 5 illustrates a conceptual view of a game play screen according to an embodiment of the present invention.

The metronome 501 is an example of the respiration inducing indicator. The metronome 501 expresses the period of the respiration waveform template. For example, when the period of the respiration waveform template is 10 seconds, a number of an activation bar of the metronome may be increased over 5 seconds and a number of the activation bar may be decreased over 5 seconds.

On the other hand, the controller 330 may gradually change a period of the metronome 501. For example, it is assumed that the period of the respiration waveform data is 5 seconds and the period of the respiration waveform template is 10 seconds. In other words, the ideal respiration status is relatively slow whereas the current respiration status of the user, to the contrary, is fast. Accordingly, the controller 330 may not fix the period of the metronome 501 to be 10 seconds in an early stage but may gradually change the period of the metronome 501 from 5 seconds, which is the period of the respiration waveform data, to 10 seconds. Therefore, an effect such that the user naturally reaches the ideal respiration status may be created.

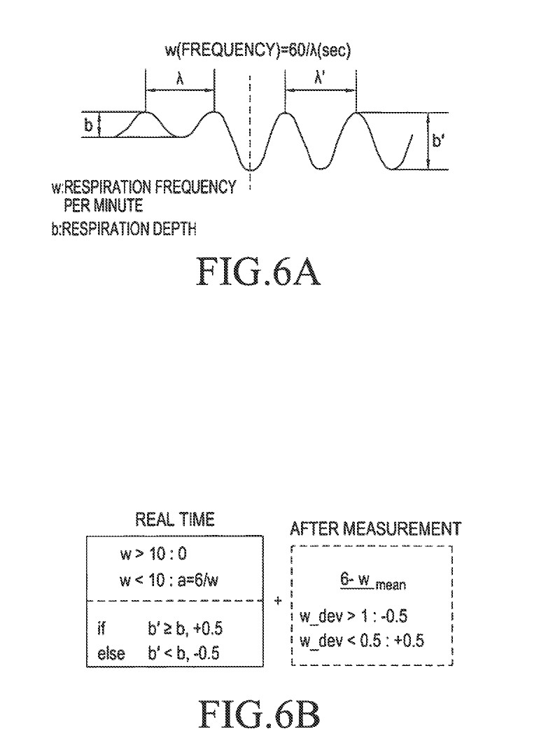

An acquired coin number 502 represents the respiration fidelity based on the similarity between the respiration waveform data and the respiration waveform template in the form of a user-friendly score. A total acquired score 503 represents a final result according to counting the acquired coin number 502. The acquired coin number and the score will be described below in more detail with reference to FIGS. 6A and 6B.

A fixed location 505 is a location at which the character exists. In a game according to an embodiment of the present invention, the character is located at the fixed location 505 and moves in an upward and downward direction 506 without movement in a left and right direction. The character moves in the upward and downward direction 506 according to a rise and fall of a topographical object 508. For example, a character may be displayed as if disposed on the topographical object 508.

On the other hand, the topographical object 508 may correspond to the respiration waveform data and a topographical object 509 may correspond to the respiration waveform template. Also, the topographical objects 508 and 509 may move in a preset direction 507. Namely, the topographical objects 508 and 509 move in a specific direction as time elapses, but the character does not move in the left and right direction, only up and down, thereby creating an effect as if the character moves on the topographical object 508.

A plurality of coins 504 may be disposed on the topographical object 509 corresponding to the respiration waveform template. In other words, when the user outputs the respiration waveform data similar to the respiration waveform template, the character obtains a coin.

FIGS. 6A and 6B illustrates conceptual views for explaining the respiration fidelity and a score thereof. As shown in FIG. 6A, the respiration waveform data includes a period λ and a depth b in an early stage, and includes a period λ′ and a depth b′ in a later stage. On the other hand, it is assumed that the period λ corresponds to a frequency of w and the period λ′ corresponds to a frequency of w′.

FIG. 6B illustrates an evaluation criteria for a user's respiration. A part shown in real time is a score counted during game play, and a part shown after measurement means a score counted after the game play. In a case where the frequency w of the current respiration waveform data exceeds 10, a score is not assigned. On the other hand, in a case where the frequency w is below 10, a result of dividing 6 by the frequency w is assigned as a score (a).

Also, if a subsequent depth b′ is deeper than a previous depth b, a score of 0.5 is assigned, and if the previous depth b is deeper than the subsequent depth b′, a score of −0.5 is assigned.

On the other hand, after the game play is finished, when a value w_dev obtained by subtracting an average frequency wₘₑₐₙ from 6 is greater than 1, the score of −0.5 is assigned, and when the value w_dev obtained by subtracting the average frequency wₘₑₐₙ from 6 is less than 0.5, the score of 0.5 is assigned.

Further, in real time, a graphic may be rendered such that a gold coin (representing 5 won in Korean currency or 5 dollars in US currency) is obtained in a case of a score greater than 3 points, a silver coin (representing 2 won in Korean currency or 2 dollars in US currency) is obtained in a case of a score between 2 points and the 3 points, and a copper coin (representing 1 won in Korean currency or 1 dollar in US currency) is obtained in a case of a score between a 1 point and the 2 points.

According to the game play described above, the user may adjust, with interest, the respiration such that the user's respiration waveform data approximates the respiration waveform template.

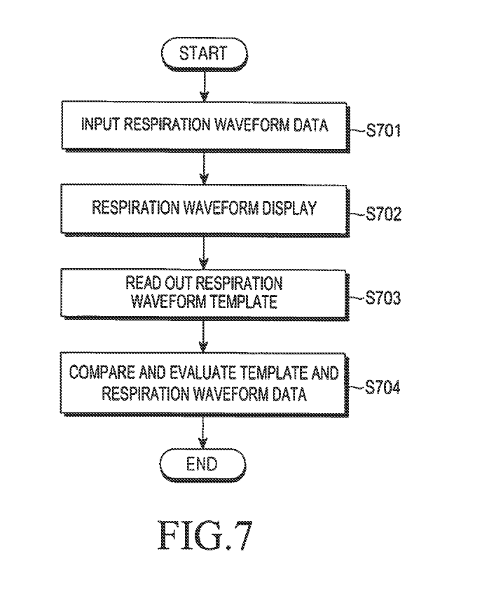

FIG. 7 illustrates a flow chart of a control method of a respiration inducing apparatus according to another embodiment of the present invention.

The respiration inducing apparatus receives the respiration waveform data at step S701. Here, the respiration inducing apparatus is a means for receiving the respiration waveform data and includes a three axis acceleration sensor. The respiration inducing apparatus may receive the respiration waveform data based on various methods other than sensing the acceleration, which will be described below in further detail.

The respiration inducing apparatus displays the respiration waveform data at step S702. The user identifies the user's respiration status by monitoring the respiration waveform data in real time. Further, the respiration inducing apparatus reads out the respiration waveform template in step S703.

The respiration inducing apparatus estimates the respiration fidelity about the current respiration status by comparing the input respiration waveform data with the read out respiration waveform template at step S704. The respiration inducing apparatus may, for example, determine the similarity between the respiration waveform data and the respiration waveform template based on the correlation which is related to Equation (1). The respiration inducing apparatus may display the determined similarity, i.e., respiration fidelity. As a result, the respiration inducing apparatus displays at least one of the respiration waveform data, the respiration waveform template, and the respiration fidelity, thereby creating an effect of naturally inducing the ideal respiration status from the user.

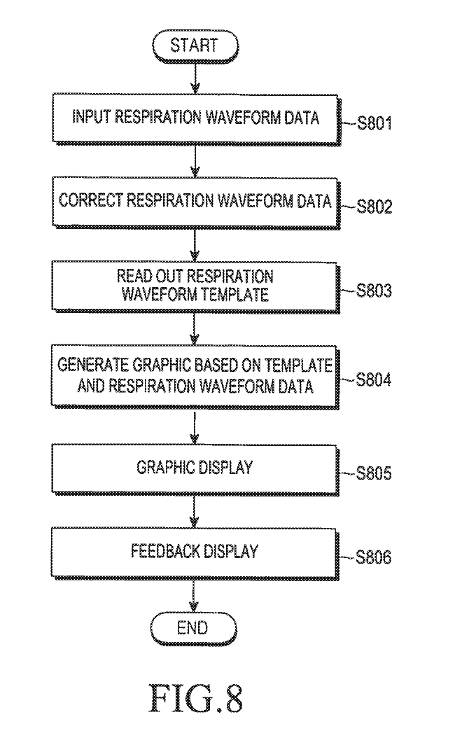

FIG. 8 illustrates a flow chart of a control method of a respiration inducing apparatus according to another embodiment of the present invention.

The respiration inducing apparatus receives the respiration waveform data at step S801. The respiration inducing apparatus performs correction on input respiration waveform data at step S802. The respiration inducing apparatus may, for example, amplify the input respiration waveform data with the preset gain and may apply at least one filtering process among a low pass filtering, a high pass filtering, and a band pass filtering to the amplified respiration waveform data.

The respiration inducing apparatus reads out the respiration waveform template at step S803. Also, the respiration inducing apparatus renders a graphic based on the respiration waveform data and the respiration waveform template at step S804. Here, the graphic may be one screen of a game screen which may trigger the user's interest. The respiration inducing apparatus displays the rendered graphic at step S805 and the user may, for example, perform game play corresponding thereto.

The respiration inducing apparatus also receives feedback respiration waveform data at step S806, which is changed respiration waveform data output by the user when the user recognizes the graphic and renders and displays the feedback respiration waveform data. Accordingly, an effect of allowing the user to identify a changed circumstance of the respiration status of the user may be created.

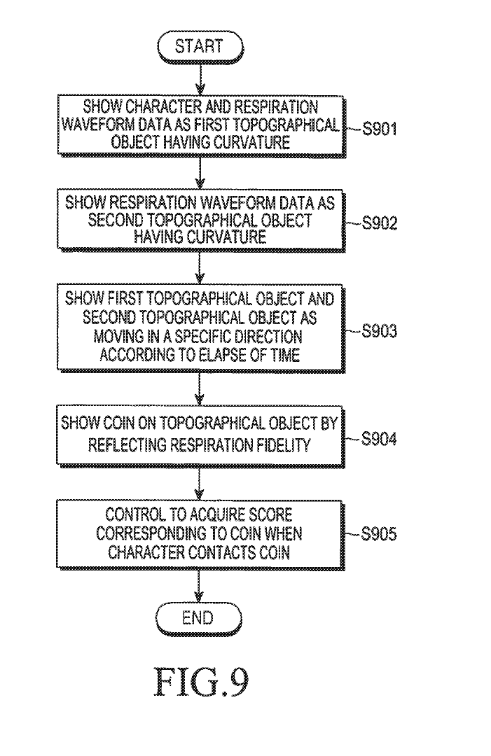

FIG. 9 illustrates a flow chart of a game screen display method according to another embodiment of the present invention.

The respiration inducing apparatus shows the character and also shows the input respiration waveform data as a first topographical object having a curvature at step S901.

Also, the respiration inducing apparatus shows the respiration waveform template as a second topographical object having a curvature at step S902. The respiration inducing apparatus shows the first topographical object and the second topographical object moving in a specific direction as time elapses at step S903. In this case, the character may be controlled not to move in the left and right direction but only move in the upward and downward direction, thereby creating an effect that the character is shown as moving on the first and the second topographical objects.

The respiration inducing apparatus shows a coin reflecting the respiration fidelity on the second topographical object at step S904. Here, the type of the coin and the score corresponding thereto have already been described in detail, and thus, a further description thereof will be omitted.

When the character contacts the coin, the respiration inducing apparatus controls to acquire a score or a number corresponding to the coin at step S905. According to the above described process, the user may adjust the respiration status such that the respiration waveform data approximates the respiration waveform template in order for the character to acquire more coins or a higher score.

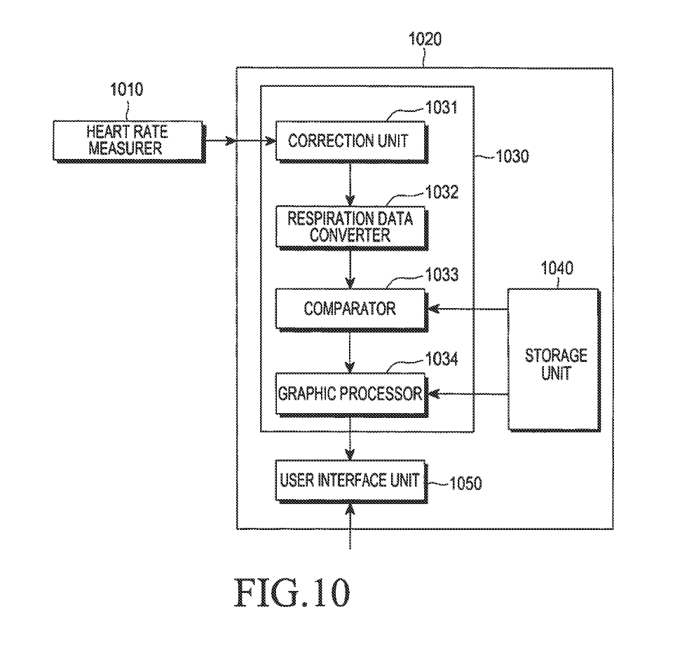

FIG. 10 illustrates a block diagram of a respiration inducing apparatus according to another embodiment of the present invention.

As shown in FIG. 10, a respiration inducing apparatus 1020 is connected to a heart rate measurer 1010. The respiration inducing apparatus 1020 includes a controller 1030, a storage unit 1040, and a user interface unit 1050. The controller 1030 includes a correction unit 1031, a respiration data converter 1032, a comparator 1033, and a graphic processor 1034.

The respiration inducing apparatus 1020 according to the embodiment of FIG. 10 induces the respiration waveform data based on an input heart rate rather than directly collecting the respiration waveform data.

The heart rate measurer 1010 measures a heart rate of the user during a preset period. The heart rate measurer 1020 includes, for example, an electrode or a light sensor. The heart rate measurer 1020 may be mounted to a chest, an arm, or a leg of the user when the heart rate measurer 1020 is implemented as an electrode and may be mounted near an arterial blood vessel or a venous blood vessel when the heart rate measurer 1020 is implemented as an light sensor.

The heart rate measurer 1010 outputs the heart rate of the user to the correction unit 1031. The correction unit 1031 performs at least one of amplifying or filtering of input heart rate data.

The respiration data converter 1032 converts the input heart rate data into the respiration waveform data. The respiration data converter 1032 converts heart rate data into the respiration waveform data based on a relationship between the heart rate and the respiration waveform. For example, it is assumed that the heart rate and the respiration waveform have a relationship such that the period of the respiration waveform data is decreased when the heart rate is increased and the period of the respiration waveform data is increased when the heart rate is decreased. In this case, the respiration data converter 1032 generates the respiration waveform data by considering a change of the heart rate data.

A step of the comparator 1033 comparing the converted respiration waveform data and the respiration waveform template read out from the storage unit 1040, a step of the graphic processor 1034 rendering a graphic, and a step of the user interface unit 1050 displaying the rendered graphic or receiving the user input have already described, and thus, a detailed description thereof will be omitted.

In the embodiment of FIG. 10, it is shown that the heart rate measurer 1010 is connected to the respiration inducing apparatus 1020; however, those skilled in the art would understand that the present invention is not limited to this as long as an apparatus may measure data which can be converted into the respiration waveform data. For example, those skilled in the art may easily devise embodiments in which a brainwave sensor, a skin resistance sensor, a skin temperature sensor, or a muscle signal sensor is connected to the respiration inducing apparatus 1020 in replacement of the heart rate measurer.

Accordingly, a structure in which various biometric signals are measured and converted into the respiration waveform data to induce respiration is possible, and a user may use a biometric signal from a desired part, thereby maximizing user convenience.

While the present invention has been shown and described with reference to certain embodiments thereof, it will be understood by those skilled in the art that various changes in form and details may be made therein without departing from the spirit and scope of the present invention as defined by the appended claims.

What is claimed is:

1. A respiration inducing apparatus, comprising:
a respiration waveform data collection unit for collecting respiration waveform data from a user;
a storage unit for storing a respiration waveform template;
a controller for comparing the respiration waveform data and the respiration waveform template and determining a respiration fidelity based on a similarity of a comparison result; and
a user interface unit for displaying at least one of the respiration waveform data, the respiration waveform template, and the respiration fidelity.

2. The respiration inducing apparatus of claim 1, wherein the respiration waveform data collection unit comprises an acceleration sensor for sensing an acceleration applied to the respiration inducing apparatus.

3. The respiration inducing apparatus of claim 2, wherein the controller interprets the respiration waveform data based on acceleration information from the respiration waveform data collection unit.

4. The respiration inducing apparatus of claim 1, wherein the controller determines the respiration fidelity based on a correlation between the respiration waveform data and the respiration waveform template.

5. The respiration inducing apparatus of claim 4, wherein the respiration waveform data is an n number of time series data, the respiration waveform template is an n number of time series data, and the correlation is determined by the following:
$${r}_{xy} = \frac{{\sum }_{i = 1}^{n} ( {x}_{i} - \overline{x} ) ( {y}_{i} - \overline{y} ) }{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}}$$
, wherein x̅ is an average of the respiration waveform data y̅ and is an average of the respiration waveform template.

6. The respiration inducing apparatus of claim 1, wherein the respiration fidelity is represented as a score through playing of a preset game, and the controller renders the preset game and controls the user interface unit to display a rendered game screen.

7. The respiration inducing apparatus of claim 6, wherein, when a depth of the respiration waveform data is increased time serially, the controller treats a result of adding an additional score to the score as a score.

8. The respiration inducing apparatus of claim 6, wherein the controller compares an average of a frequency of the respiration waveform data and a frequency of the respiration waveform template to calculate the score.

9. The respiration inducing apparatus of claim 1, wherein the respiration waveform data collection unit comprises:
a correction unit for receiving and correcting a biometric signal; and
a respiration data converter for converting the biometric signal into the respiration waveform data.

10. The respiration inducing apparatus of claim 9, wherein the biometric signal is heart rate data.

11. A method of controlling a respiration inducing apparatus, comprising:
collecting respiration waveform data from a user;
reading out a pre-stored respiration waveform template;
comparing the respiration waveform data and the respiration waveform template and determining a respiration fidelity based on a similarity of a comparison result; and
displaying at least one of the respiration waveform data, the respiration waveform template, and the respiration fidelity.

12. The method of claim 11, wherein collecting the respiration waveform data comprises:
sensing an acceleration applied to the respiration inducing apparatus.

13. The method of claim 12, further comprising:
interpreting the respiration waveform data based on acceleration information.

14. The method of claim 11, wherein determining the respiration fidelity comprises:
determining the respiration fidelity based on a correlation between the respiration waveform data and the respiration waveform template.

15. The method of claim 14, wherein the respiration waveform data is an n number of time series data, the respiration waveform template is an n number of time series data, and the correlation is determined by the following:
$${r}_{xy} = \frac{{\sum }_{i = 1}^{n} ( {x}_{i} - \overline{x} ) ( {y}_{i} - \overline{y} ) }{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}}$$
, wherein x̅ is an average of the respiration waveform data and y̅ is an average of the respiration waveform template.

16. The method of claim 11, wherein the respiration fidelity is represented as a score through playing of a preset game, and the method further comprises:
rendering the preset game; and
controlling a user interface unit to display a rendered game screen.

17. The method of claim 16, wherein, when a depth of the respiration waveform data is increased time serially, the controller treats a result of adding an additional score to the score as a score.

18. The method of claim 16, wherein the controller compares an average of a frequency of the respiration waveform data and a frequency of the respiration waveform template to calculate the score.

19. The method of claim 11, wherein collecting the respiration waveform data comprises:
receiving and correcting a biometric signal; and
converting the biometric signal into the respiration waveform data.

20. The method of claim 19, wherein the biometric signal is heart rate data.
